(19) United States
(12) Patent Application Publication
(10) Pub. No.: US 2004/0193446 A1
(43) Pub. Date: Sep. 30, 2004

(54) System and method for managing a patient treatment program including a prescribed drug regimen

(76) Inventors: Steven Lloyd Mayer, Salem, WI (US); John Urquhart, Palo Alto, CA (US); Jean-Michel Metry, Sion (CH); Bernard Vrijens, Eben Emael (BE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/400,271

(22) Filed: Mar. 27, 2003

(51) Int. Cl. G01N 33/50

(52) U.S. Cl. 705/2; 221/2; 702/19; 600/300

(57) ABSTRACT

The present invention relates to a system and method for managing a patient treatment program including a prescribed dosing regimen. The system and method develops and/or makes use of a pharmacokinetic model and a pharmacodynamic model and the monitored adherence of the patient to determine if and when testing should be performed. The system and method further determines if the prescribed dosing regimen should be adjusted, based upon a comparison of the results of the one or more tests and the results predicted by the one or more models.

### FIELD OF THE INVENTION

The invention pertains to a system and method for managing patient care associated with a prescribed drug regimen including predictive models used in combination with monitored compliance and testing.

### BACKGROUND OF THE INVENTION

Prescribed drugs can only be effective if properly taken. For many drugs there is often a finite usage range in which the drugs will produce the intended results. If not enough of a drug is taken, a drug may only be partially effective, may be non-effective, and/or may even promote undesirable effects. If too much of a drug is taken, undesirable side effects of the drug may manifest or become more pronounced.

One of the goals when prescribing medication in the treatment of a patient is to determine the proper amount of a drug, and the corresponding dosing interval, to produce the desired effect. However prescribing a proper amount of a drug and the related proper dosing interval is just part of the story. The patient then needs to take the medication as prescribed.

Many studies suggest that poor and partial adherence of patients to a prescribed drug regiment is prevalent, and many studies show that 50 percent or more of all patients prescribed drugs do not take them as prescribed. In a further study, of the number of doses prescribed, generally, one-third of the patients took greater than 95 percent of the prescribed doses, another third of the patients took between 70 and 95 percent of the prescribed doses, and the final third of the patients took fewer than 70 percent of the prescribed doses. Results indicative of poor or partial adherence are found even with life-saving treatment regimens, e.g., antiretroviral drug regimens prescribed in the treatment of an HIV infection. Poor and partial adherence for prescribed drug regimens also prevails to varying degrees for other types of chronic diseases or conditions, such as thyroid disease, hypertension, congestive heart failure, epilepsy, obesity and cancer.

Not only can patient compliance be a problem, but recognition of poor and partial compliance, in some instances, can go undetected by a care giver. In these instances, a poor response to a prescribed drug regimen can sometimes be falsely attributed to an inadequacy in the drug regimen. This in turn may prompt unnecessary changes to be made to the prescribed drug regimen, where sometimes the type or combination of drugs prescribed and/or the dosage levels may be altered. In some instances this may prompt a change in a drug regimen, which would otherwise have been effective, had proper compliance been maintained.

In an effort to detect poor compliance, and therefore minimize unnecessary changes, some care givers have instituted compliance monitoring as part of a prescribed drug regimen. One such approach includes tracking the number of doses taken during a prescribed period and comparing the number against the number of doses prescribed. However in using such an approach, an extra dose taken during one period can mask a missed dose in another period. Furthermore such an approach also fails to identify doses taken at the wrong time, where the doses may have been taken too late, whereby a longer period between doses occurs than was otherwise intended.

Because the drugs prescribed often have a relatively short half life, which relates to the time that the drug remains present in the patient's plasma and the corresponding concentration of the drug over time, large delays between doses and/or missed doses can create periods in which the drug concentration in the patient's plasma falls below levels needed for effective therapeutic action of the drug in question. In terms of the treatment of a viral infection, like HIV, ineffective concentrations, in addition to impacting the ability of the drug to suppress the virus, may create selection pressure, that encourages the emergence of a drug resistant strain. This undesirable situation occurs because drug levels, which promote only partial suppression, will generally have a greater impact on a strain of the virus that is non-resistant, as opposed to a strain of the virus that is more resistant to medication. Greater suppression of the non-resistant strain will allow a resistant strain to emerge and become dominant.

In an effort to more closely track patient compliance, monitoring systems have been developed, which not only track the number of doses taken over a predetermined period of time, but also keep track of the day and time each of the doses has been taken. One such system is the MEMS® monitor produced by AARDEX Ltd. At least one version of the MEMS® monitor includes a cap closure adapted with sensors which detect the removal and the subsequent re-attachment of the cap from an enclosure containing the medication, and circuitry for recording the time and date when the cap is removed and re-attached. It is assumed that during each removal/re-attachment of the cap, a single dose of medication is dispensed from the enclosure and taken by the patient.

The monitored usage information can then be used in conjunction with predetermined characteristics of the prescribed medication, as well as the results of patient testing to make decisions concerning possible alterations in the patient's drug treatment program so as to provide safe and effective care.

However there is a cost associated with each activity, including a cost of the various tests to monitor the patient's condition, as well as a cost to performing the monitoring. Furthermore, the accepted characteristics of the prescribed medication, often relate to determined averages, some of which may or may not directly apply to a particular patient. Still further, given the number of variables involved, in monitoring, predicting, testing and interpreting the effects of the current prescribed dosing regimen, decisions concerning the need for adjustments in a patient's prescribed dosing regimen can be quite complex. This is further complicated by a desire to manage the patient's care, in a manner which is cost effective.

Consequently, it would be beneficial to develop a system and method for managing patient care associated with a prescribed drug regimen including predictive models used in combination with monitored compliance and testing. In at least some instances it would be beneficial to be able to individualize the predictive models and to be able to determine or confirm the accuracy of the models, as they relate to a particular patient, by correlating the predicted results with the measured response determined through testing, and to determine if and when testing should be performed for producing useful results.

Still further, it would similarly be beneficial to be able to determine a drug's effectiveness in producing a desired pharmacological effect over a broad range of patient adherence for determining the expected varying pharmacological impact of the drug as a function of change in adherence.

### SUMMARY OF THE INVENTION

A method of individualizing the treatment of a patient associated with a prescribed drug regimen is provided. The method provides for the development of a pharmacokinetic model, which predicts the drug concentration over time in the patient in response to the drug dosage history of the patient, and the development of a pharmacodynamic model, which includes a predicted level of effectiveness for various levels of dosing and various degrees of deviation from the prescribed dosing regimen. A drug regimen is then prescribed for the patient, designed to achieve a desired pharmacological effect, based upon the pharmacokinetic model and the pharmacodynamic model.

The patient is then monitored to determine a degree of deviation from a prescribed dosing regimen. The pharmacological effect in the patient of the prescribed dosing regimen is then measured, and compared with the level of effectiveness that was predicted by the pharmacodynamic model, after taking into account the adherence of the patient to the prescribed dosing regimen.

If the measured effect deviates from the predicted level of effectiveness, the method then provides for additional tests to be performed to determine the actual drug concentration over time in the particular patient. The pharmacokinetic model is then adjusted based upon the actual determined drug concentration over time, and the prescribed drug regimen is adjusted for the patient to account for adjustments in the pharmacokinetic model.

In a further embodiment, an economic model is used to determine the most cost effective course in correcting for non-compliant patient behavior, if any. The economic model similarly enables the cost of the test to be compared against the likelihood of producing meaningful information, which can be used in verifying and adjusting the patient's present care, and in determining the order in which tests should be performed.

In yet a further embodiment, a method is provided for designing a clinical trial, which determines the effectiveness of a drug in producing a desired pharmacological effect over a broad range of patient adherence to a prescribed drug regimen, where the monitored adherence of one or more patients to the prescribed drug regimen, and the measured pharmacological effect in the one or more patients at various intervals, are related based upon the degree of deviation from the prescribed dosing regimen.

In still a further embodiment of the present invention, the methods and models are implemented as part of a system including a patient health management computer program comprising a communication unit for receiving access information indicative of patient compliance. The system further includes a processor for executing a plurality of prestored instructions, corresponding to creating and maintaining a pharmacokinetic model, a pharmacodynamic model and a decision analytic model. The system also includes an interface unit for communicating with a user the type and timing of tests recommended to be performed and for receiving the results of the tests.

Numerous other advantages and features of the present invention will become readily apparent from the following detailed description of the invention and the embodiments thereof, from the claims and from the accompanying drawings.

### BRIEF DESCRIPTION OF THE DRAWINGS

FIG. 1 is an example of a graph illustrating predicted drug concentration over time of the type that would be produced by a pharmacokinetic model;

FIG. 2 is an example of a graph illustrating predicted viral suppression as a function of drug concentration of the type that would be produced by a pharmacodynamic model (confidential information communicated to Abbott by AARDEX Ltd);

FIG. 3 is an example of a graph illustrating the likelihood of change in the condition of the patient, both positive and negative, based upon the degree of adherence to a prescribed drug regimen and the preceding condition of the patient (confidential information communicated to Abbott by AARDEX Ltd);

FIG. 4 is a block diagram illustrating a model for use in managing a patient treatment program in accordance with at least one embodiment of the present invention;

FIG. 5 depicts an exemplary flow diagram of a method for individualizing the treatment of a patient associated with a prescribed drug regimen, for use with a model of the type illustrated in FIG. 4;

FIG. 6 depicts an exemplary flow diagram of a method for providing patient care, and for achieving and maintaining a level of wellness, for use with a model of the type illustrated in FIG. 4;

FIG. 7 depicts an exemplary flow diagram of the steps associated with determining the need for change in the drug regimen provided for in FIG. 6;

FIG. 8 depicts an exemplary flow diagram of a method for designing a clinical trial, which determines the effectiveness of a drug in producing a desired pharmacological effect over a broad range of patient adherence to a prescribed drug regimen, for use with a model of the type illustrated in FIG. 4;

FIG. 9 depicts an exemplary flow diagram of a method for managing an antiretroviral treatment program of a patient, for use with a model of the type illustrated in FIG. 4; and

FIG. 10 is a block diagram of one embodiment of a system for managing a patient treatment program on which at least portions of the model, illustrated in FIG. 4, and at least portions of the methods, illustrated in FIGS. 5-9, can be performed.

### DETAILED DESCRIPTION OF THE PREFERRED EMBODIMENTS

While the present invention is susceptible of embodiment in many different forms, there are shown in the drawings and will be described herein in detail specific embodiments thereof with the understanding that the present disclosure is to be considered as an exemplification of the principles of the invention and is not intended to limit the invention to the specific embodiments illustrated.

The herein described system and method are well suited for managing a patient having a chronic disease or condition. One such condition for which the present system and method are particularly well suited is the management of a patient having an HIV infection. One such system and method of treating an HIV infection includes the use of one or more protease inhibitors for suppressing the virus, or in other word inhibiting the replication of the virus. While the present system and method are also applicable in managing the treatment of a patient having other types of chronic diseases or conditions, at times, the following description makes specific reference to an example including the use of an antiretroviral drug regimen in the treatment o f an HIV infection, which is presently viewed as corresponding to and illustrative of the preferred embodiment.

A chronic disease is broadly defined as an illness that is prolonged, does not resolve spontaneously, is rarely cured completely, and requires persistent administration of one or more prescription drugs to maintain the patient in a preferred medical status. Specific examples of other types of chronic diseases and conditions, in addition to HIV, for which the present invention has been identified as being particularly applicable include thyroid disease, hypertension, congestive heart failure, epilepsy, obesity and cancer. For example, in the treatment of thyroid disease, patient compliance with the administration of thyroxine or tri-iodothyronine may be managed; in the treatment of hypertension, patient compliance with the administration of diuretics, and antihypertensive agents such as trandolapril, captopril, enalapril, betaxolol, propranolol, atenolol, metoprolol, nifedipine, verapamil, diltiazem, hydrochlorothiazide, and the like may be managed; in the treatment of congestive heart failure, patient compliance with the administration of furosemide, digoxin, potassium salts, and others may be managed; in the treatment of obesity, patient compliance with the administration of sibutramine maybe managed; and in the treatment of cancer, patient compliance with the administration of tamoxifen and other agents designed for administration by patients may be managed.

FIG. 1 illustrates a graph 10 depicting predicted drug concentration as a function of time of the type that would be produced by a pharmacokinetic model. Generally, the model depicts a prescribed medication being taken at periodic intervals during which the concentration levels 12 of the drug in the plasma of the patient changes over time. The drug levels are typically initially boosted 14 shortly after a dose 16 is taken by the patient, and then gradually declines 18 up until the time proximate to the patient taking the next dose 20.

The specific drug levels are affected by the rate at which the drug is absorbed, distributed, metabolized, and excreted by a patient. In practice the actual rate at which the drug is absorbed, distributed, metabolized, and excreted, can vary between patients. At least initially, a model will generally be reflective of the expected average across all patients. Consequently, if one or more of the specific parameters for a particular patient vary sufficiently away from the average, either separately or in combination, the model based upon the average may not be reflective of the actual drug behavior in the particular patient. Correspondingly, the model may need to be adjusted for a particular patient.

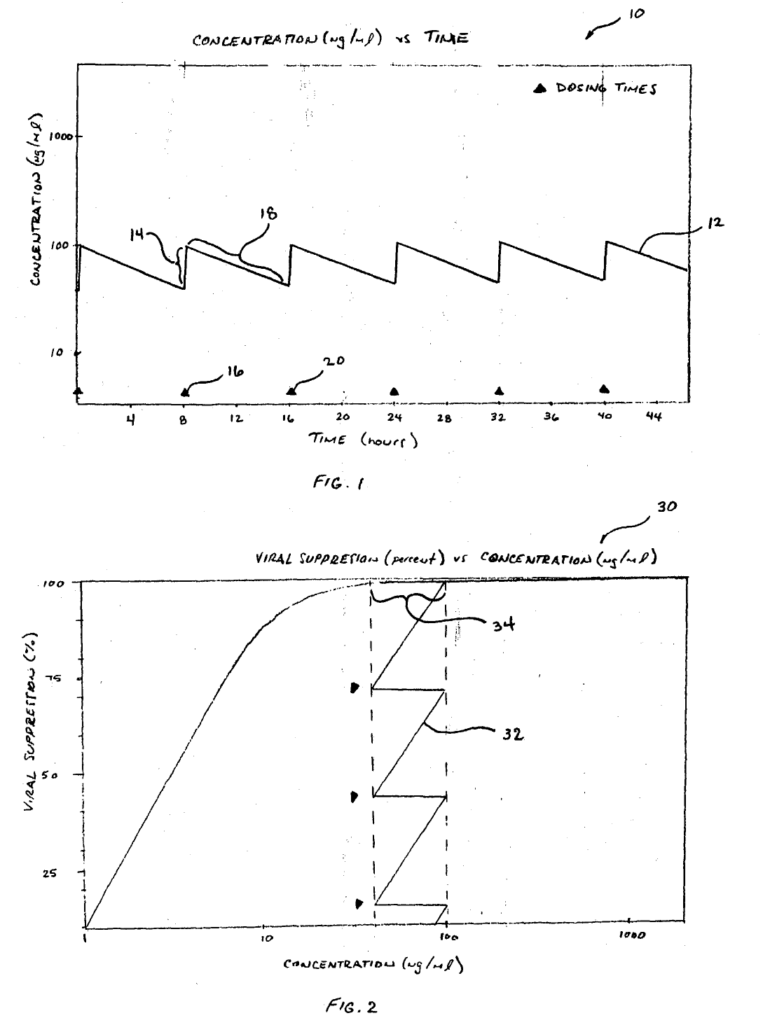

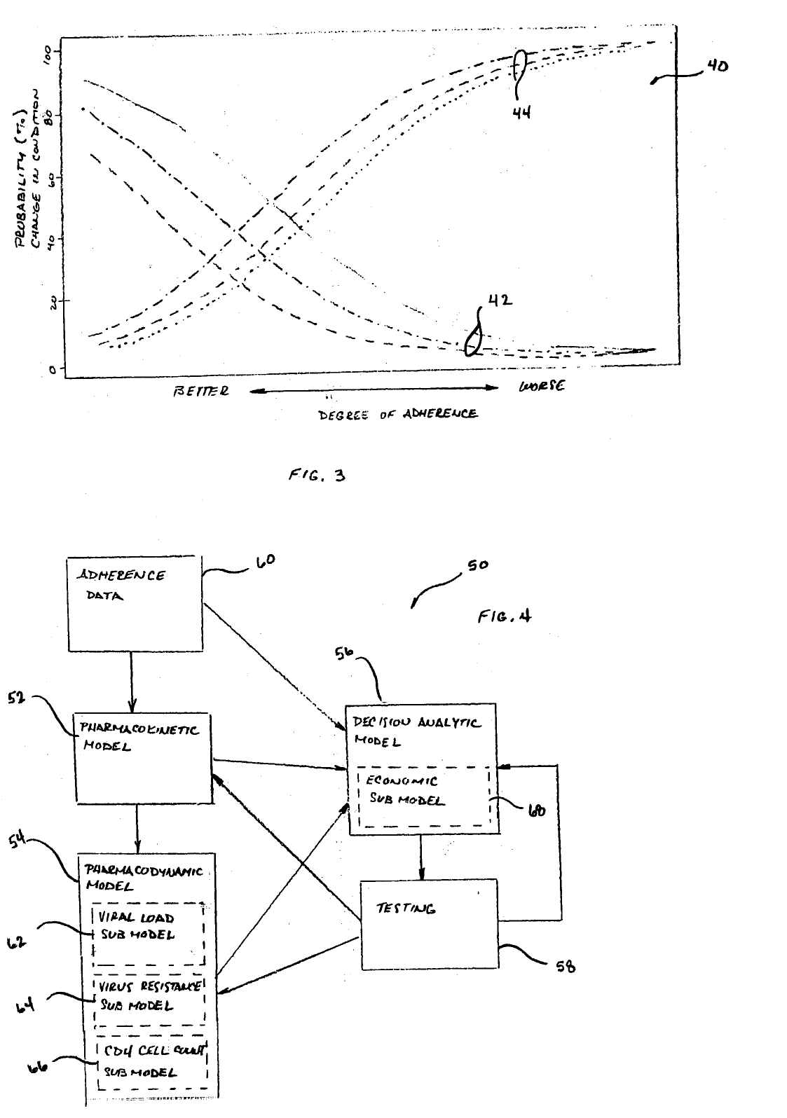

As concentration of the drug varies in the plasma (and sometimes in other fluids and tissues), often times the drug's effectiveness similarly varies. A pharmacodynamic model is intended to express the relationship between drug concentrations in the patient and a resulting pharmacological effect. One such pharmacological effect related to drug concentration is illustrated in FIG. 2.

FIG. 2 illustrates a graph 30 depicting viral suppression or inhibition of replication of a virus as a percentage for one type of protease inhibitor as against at least one strain of the virus. Generally, as the concentration of the drug increases, the drugs effectiveness in inhibiting replication similarly increases. Overlayed upon the graph 30 is the data corresponding to drug concentration levels 32, illustrated in FIG. 1. Over the anticipated range 34 of drug concentration levels, the drug effectiveness varies between approximately 97-99 percent. However one notices that if concentrations were allowed to fall further, that the decrease in effectiveness begins to accelerate.

Different strains of the virus can experience different levels of impact from varying levels of drug concentration for a particular drug. In connection with treating HIV infections, one area of concern is the emergence of drug resistant strains, that can result from suboptimal levels of treatment. At some drug concentrations, a drug may continue to be very effective against the non-drug resistant version of the strain, but begin to experience a substantial drop-off in effectiveness against resistant strains of the virus. In these circumstances the likelihood of a drug resistant strain emerging becomes more likely.

Ideally, the prescribed drug regimen is designed to provide drug concentrations that are substantially effective against both resistant and non-resistant strains. However the difficulty arises when individual or multiple doses of the prescribed drug regimen are missed or delayed thereby allowing the drug concentrations to dip further than intended. Under these circumstances the emergence of a drug resistant strain may become increasingly possible.

The emergence of a drug resistant strain is one of many pharmacological effects that can be modeled as part of a pharmacodynamic model. Still further it is possible using a pharmacodynamic model to track multiple pharmacological effects. Examples of additional pharmacological effects, which are incorporated as part of at least one of the preferred embodiments of the present invention include the effects of drug concentration levels on viral load, and the effects on CD4 cell counts as a result of maintaining a certain level of drug concentration, as part of the drug regimen.

As briefly noted above, missed or delayed doses can have a profound effect on the effectiveness of the drug in promoting the desired pharmacological effect. Despite FIG. 1 illustrating drug levels resulting from good patient compliance, perfect patient compliance rarely, if ever, occurs.

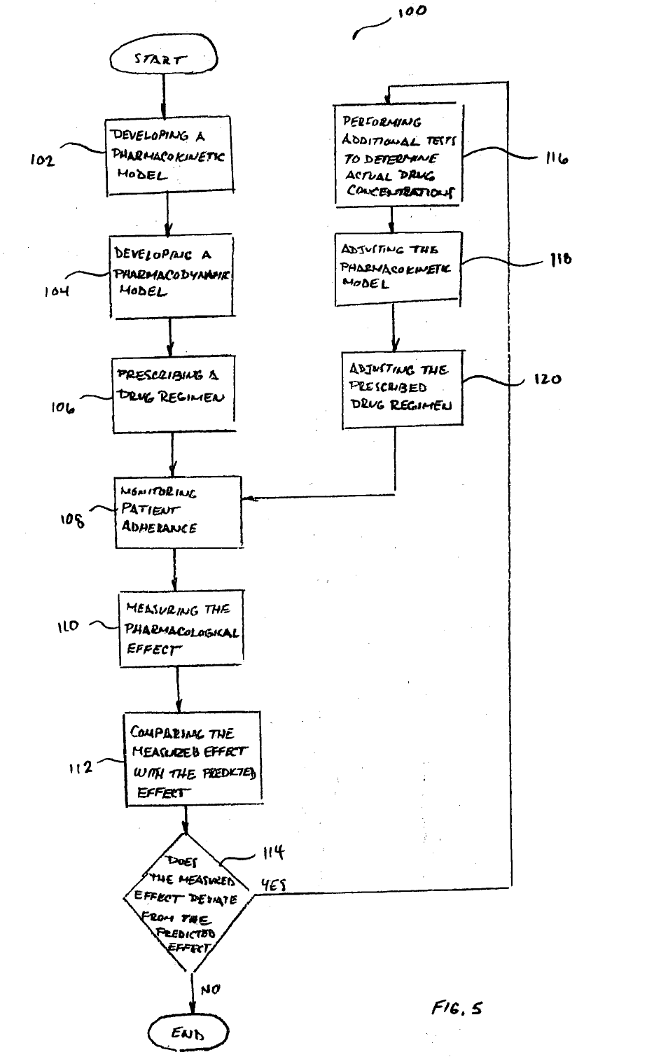

FIG. 3 illustrates a graph 40 where expected changes in a patient condition are tracked as a function of patient compliance or adherence to the prescribed drug regimen. Multiple overlaid graphs represent the likelihood of improvement and likelihood that the condition will become worse, based upon different starting conditions. As is generally the case in HIV infections, the worse the condition of the patient is when the treatment starts, the greater the opportunities to induce improvements in the patient's condition. Generally the converse is similarly true.

In graph 40 a first set of lines 42 represents the predicted likelihood that the patient's condition will improve. A second set of lines 44 represents the predicted likelihood that the patient's condition will become worse. In the case of the first set of lines 42, the top line represents a starting condition for the patient in which the viral load count is initially higher than the other two lines from the group 42. The converse is generally true with respect to the second set of lines 44, i.e. that you have a greater chance of becoming worse, if your initial condition is better.

Furthermore, the greater the deviation from optimal dosing levels the greater the likelihood of negatively impacting the chances for improvement.

Graph 40 can be used to anticipate different responses to varying levels of treatment and varying levels of compliance. An estimate as to the impact to the patient given anticipated or proposed changes in patient compliance can be quantified, which allows for cost benefit analysis to be more easily applied.

In some instances, patient compliance can be increased by intervening with the patient when non-compliance is detected. For example, explaining the consequences as to overall health of non-compliant behavior is sometimes sufficient for having an effect. Depending upon where the patient is along the curve will determine how significant of an impact a change in compliance is likely to be. At some point, the benefits may be significant enough in terms of promoting wellness that it is warranted to incur a higher degree of intervening costs. In these instances it may be worthwhile to monitor in real time the patient's dosing history, and when a delayed or missed dose is detected, page or call the patient. In other instances it may be more cost effective to adjust the patient's subsequent dosing to accommodate one or more missed doses.

By combining the multiple models and monitoring a patient's adherence to a prescribed drug regimen, the ability to develop an effective treatment program is greatly improved. In addition to being able to better predict the likely results of the treatment in the patient, the combined models can be used to predict when actual testing of the patient is likely to yield data that can be used to confirm the accuracy of the models and the corresponding effectiveness of the prescribed treatment, and/or identify other more serious issues.

FIG. 4 illustrates a model 50 for use in managing the treatment of a patient including a combination of a pharmacokinetic model 52, a pharmacodynamic model 54, and a decision analytic model 56, as well as provisions 58 for requesting that tests be performed and for receiving the test results, and provisions for receiving adherence data 60.

Generally, the adherence data 60 are received and provided to the pharmacokinetic model 52. The pharmacokinetic model 52 produces predicted drug concentrations, and supplies the same to the pharmacodynamic model 54. The pharmacodynamic model 54, produces a prediction as to one or more pharmacological effects including predictions as to viral load as part of a viral load submodel 62, the emergence of viral resistance as part of a virus resistance submodel 64, and a CD4 cell count as part of a CD4 cell count submodel 66. CD4 cell counts can be very useful in determining the likelihood of opportunistic infections, and in making the decision to prescribe additional medication to ward off the same.

All of the data are made available to the decision analytic model 56, which in turn can determine when to recommend that certain testing be performed, and can even base the decision upon rational economics using an economic submodel 68.

The results of the tests can then be used to update the model and fine tune the models to the individual patient, as well as to make determinations concerning additional recommended tests.

In at least one embodiment the combined model 50 is implemented at least in part using a computer. An example of one such system is described below in connection with FIG. 10.

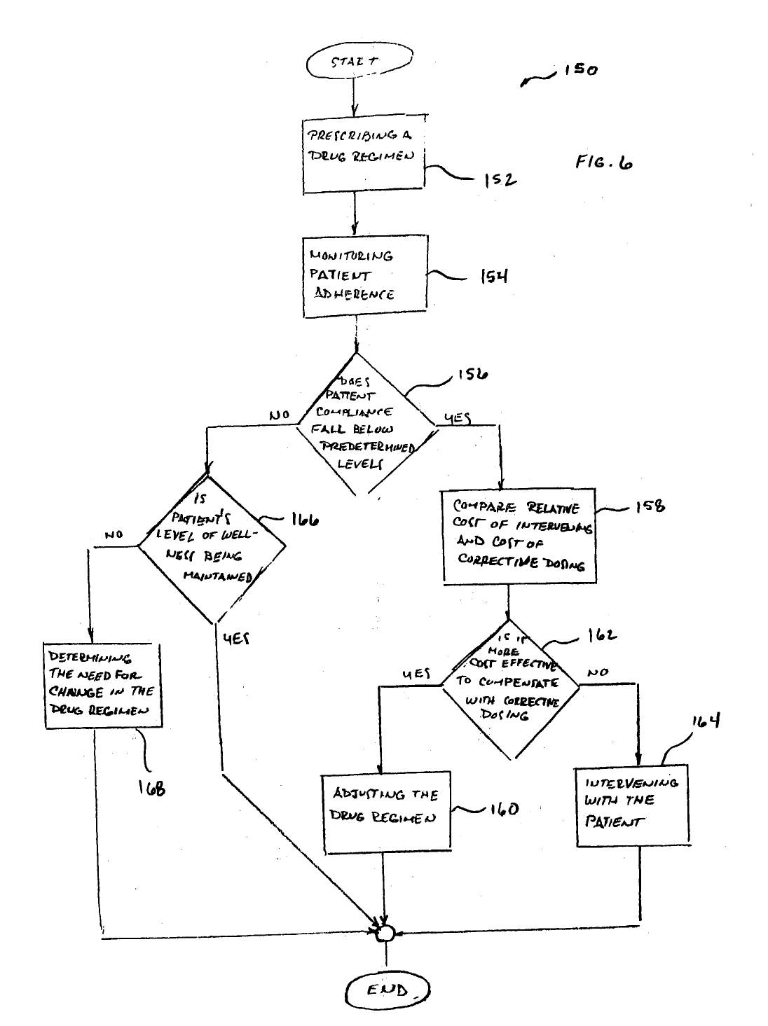

FIG. 5 depicts an exemplary flow diagram of a method 100 for individualizing the treatment of a patient associated with a prescribed drug regimen, for use with a model 50 of the type illustrated in FIG. 4. The method 100 provides for initially developing 102 both a pharmacokinetic model, which predicts the drug concentration over time in the patient in response to the drug dosage history of the patient, and developing 104 a pharmacodynamic model, which includes a predicted level of effectiveness for various levels of dosing and various degrees of deviation from a prescribed drug regimen. Generally both a pharmacokinetic model and a pharmacodynamic model can be developed as part of clinical trial. However previous clinical trials have generally not separately determined effectiveness, based upon patient adherence.

A drug regimen is then prescribed 106. The adherence to the prescribed drug regimen is then monitored 108. Testing is then performed to measure 110 the pharmacological effect of the drug dosing regimen. The measured effect is then compared 112 with the effect predicted by the pharmacodynamic model after taking into account the adherence data of the patient. Taking into account the adherence data can be important, because as noted above, the actual adherence can have a profound effect, and may be able to explain poor results.

If the measured effect deviates from the expected result 114, even after taking into account the adherence of the patient, then the method provides for performing 116 additional tests for determining actual drug concentrations. A common test for determining the actual drug concentrations is known as therapeutic drug monitoring. Such a test can determine if this particular patient is not well represented by the general pharmacokinetic model directed to the average patient.

If the test results suggest that the pharmacokinetic model fails to provide an adequate prediction for this particular patient, a determination is then made as to what changes need to be made to the pharmacokinetic model, and the adjustments are made 118. The prescribed drug regimen is then adjusted 120 accordingly. In this way, a method 100 of individualizing the treatment of a patient can be accomplished.

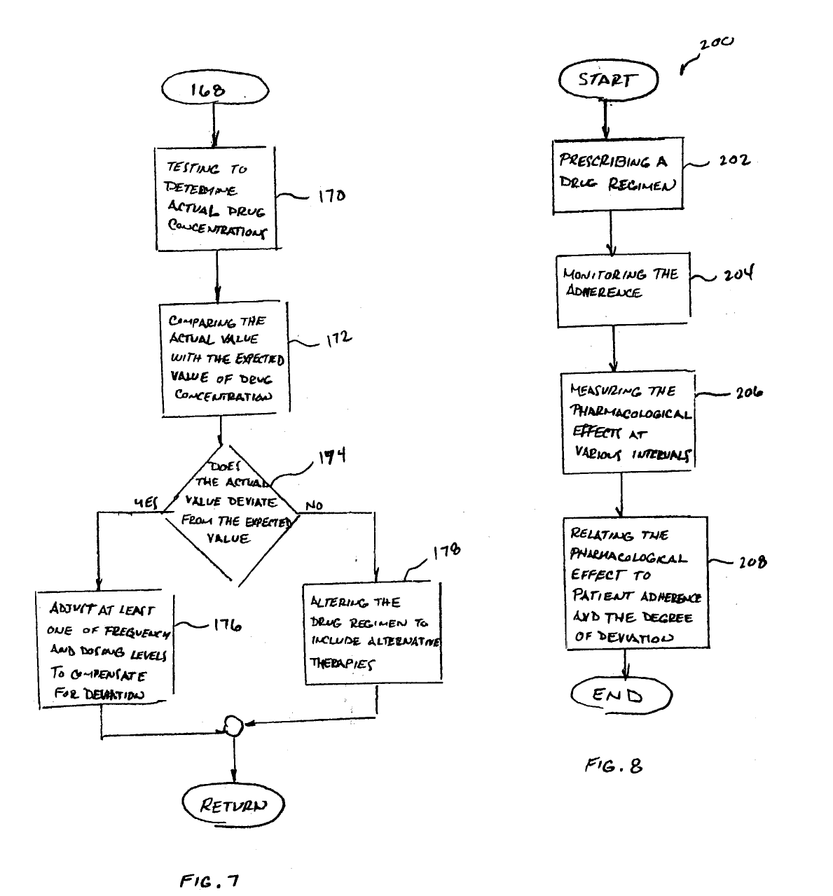

FIG. 6 depicts an exemplary flow diagram of a method 150 for providing patient care, and for achieving and maintaining a level of wellness, for use with a model of the type illustrated in FIG. 4. Initially, a drug regimen is prescribed 152, that is directed to achieving and maintaining a predetermined level of wellness. The adherence of the patient to the drug regimen is then monitored 154. A determination 156 is then made as to whether compliance levels are being maintained at satisfactory levels. If the level of compliance falls below the satisfactory levels, a determination is made of the anticipated cost to compensate for non-compliant behavior, and the cost for corrective drug dosing is compared against the cost of intervening with the patient 158.

The method then provides for adjusting the drug regimen 160, if it is determined to be more cost effective 162. Alternatively, if the available intervention alternatives are more cost effective, the method then provides for intervening with the patient 164.

As noted previously, intervening activity can include paging or calling the patient when non-compliance is detected. It can also include patient education concerning the significance and effect of non-compliance. Initially lower cost interventions can be tried and the adherence monitored to determine if the intervention was successful. Later more expensive interventions can be attempted, if necessary, and if it is estimated that they will be more cost effective than corrective dosing.

If the patient's adherence is good, but the predetermined level of wellness fails to be maintained 166, then the model determines 168 whether there is a need for a change in the prescribed drug regimen.

FIG. 7 depicts an exemplary flow diagram of the steps associated with determining the need for change in the drug regimen 168 provided for in FIG. 6. Initially, testing is performed 170 to determine the actual drug concentrations in the patient. As noted previously, sometimes the pharmacokinetic model needs to be adjusted for a particular patient. The actual level of drug concentration over time is then compared 172 against the expected values predicted by the pharmacokinetic model. If the actual drug concentration levels deviate from the expected value 174, the method then adjusts 176 at least one of the dosing frequency and dosing levels to compensate for the deviation.

If actual drug concentration levels are in line with expected drug concentration levels, then the method provides for altering 178 the drug regimen to include alternative therapies. In the case of treating an HIV infection, another drug could be prescribed for which the patient's form of the virus has not developed a resistance.

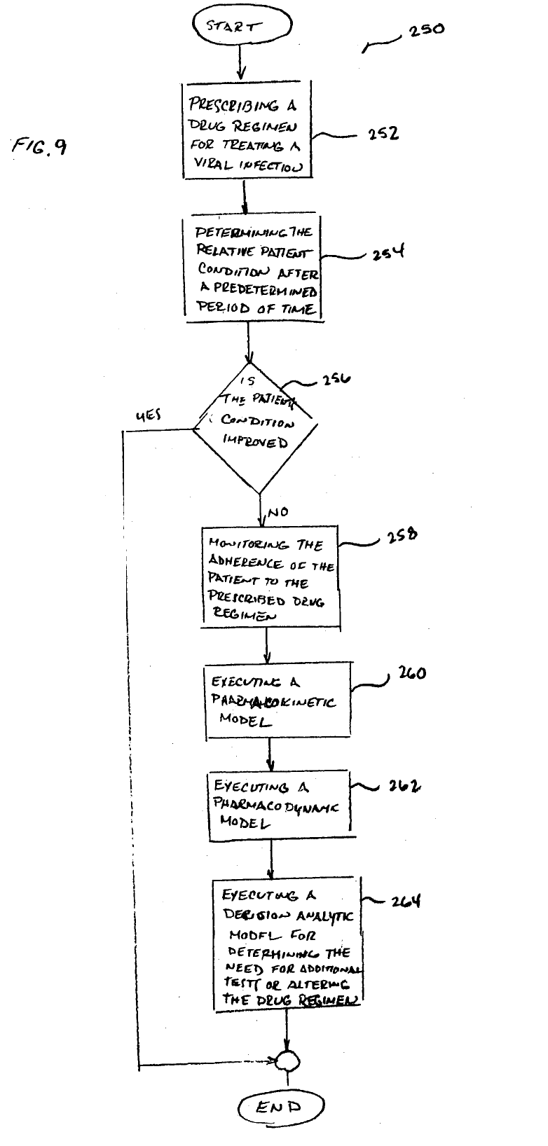

FIG. 8 depicts an exemplary flow diagram of a method 200 for designing a clinical trial, which determines the effectiveness of a drug in producing a desired pharmacological effect over a broad range of patient adherence to a prescribed drug regimen, for use with a model of the type illustrated in FIG. 4. Initially a drug regimen is prescribed 202 to one or more patients. The adherence to the prescribed drug regimen for each of the one or more patients is then monitored 204. The pharmacological effect for each of the one or more patients is then measured 206 at various intervals. The measured pharmacological effect is then related 208 to the patient adherence data for determining the pharmacological effect over a broad range of patient adherence to a prescribed drug regimen.

The method 200 benefits from the inherent variability in patient adherence, and in turn uses the resulting test data as useful information from which future results can be predicted, based upon broader ranges of adherence. Where the monitored adherence is at a level for which insufficient predictive data exists, the method could prompt the patient for additional testing.

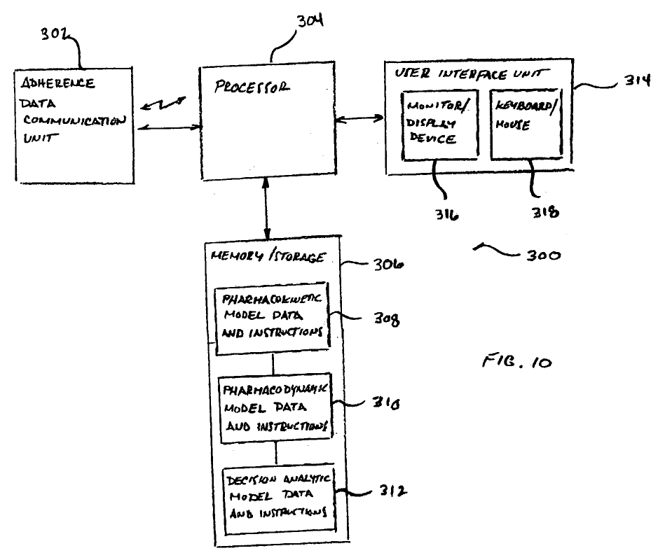

FIG. 9 depicts an exemplary flow diagram of a method 250 for managing an antiretroviral treatment program of a patient, for use with a model of the type illustrated in FIG. 4. Initially, a drug regimen is prescribed 252 for treating a viral infection. The condition of the patient is then determined after a predetermined period of time 254. If the patient's condition has improved 256, then no changes are made to the regimen.

If the patient's condition has not improved 256, then the method provides for the monitoring 258 of the adherence of the patient to the prescribed drug regimen. A pharmacokinetic model is then executed 260, in conjunction with a pharmacodynamic model 262. The method then further executes 264 a decision analytic model for determining the need for additional tests or for determining the need to alter the drug regimen.

While previously it has been noted that it may be desirable to update a pharmacokinetic model, so as to more closely correspond to a particular patient, it is also possible that the pharmacodynamic model should be updated to account for characteristics unique to the patient. Correspondingly the results of the viral resistance testing or other related testing might suggest, or make desirable, that the pharmacodynamic model be updated.

FIG. 10 is a block diagram of one embodiment of a system 300 for managing a patient treatment program on which at least portions of the model, illustrated in FIG. 4, and at least portions of the methods, illustrated in FIGS. 5-9, can be performed. The system 300 includes a patient health management computer including an adherence data communication unit 302. The communication unit 302 can take the form of several well known communication interfaces for a computer of the type including a modem, a radio transceiver, a serial or parallel interface, a SCSI adapter, a USB adapter, and network interface card. In at least one embodiment the communication unit 302 includes an interface cradle for receiving one or more of the enclosures serving as a compliance monitoring device. Alternatively the communication unit 302 could receive the data wirelessly. In at least one embodiment, the noted enclosures could take the form of the MEMSO® monitoring device discussed in the background of the art section.

The system 300 further includes a processor 304 for executing a plurality of prestored instructions. The instructions are generally stored in some form in memory, such as ROM or RAM, or as part of some auxiliary storage device, such as an optical disk, a hard disk, or a floppy disk. The memory/storage 306 in which the operating instructions and corresponding data are stored can be integral to the processor, or part of a separate connected unit.

The stored instructions and data include instructions 308 for creating and maintaining a pharmacokinetic model, instructions 310 for creating and maintaining a pharmacodynamic model, and instructions 312 for creating and maintaining a decision analytic model.

The system 300 still further includes a user interface unit 314 for communicating to a user any recommendation as to when an action should be performed related to the management of patient care. Such actions could include prompting for a test to be performed, and indications that the patient needs to be contacted concerning a reminder to take his/her medication. The communication could be displayed on a monitor or display device 316. The communication could alternatively be communicated audibly through a speaker.

The user interface unit 314 additionally enables the user to supply data to the computer. Traditionally such communication has been performed through devices such as a keyboard, a mouse or other pointing device 318. Other forms of user interface devices include touch screens, or microphones. One skilled in the art will readily recognize other forms of communication through other types of user interface devices are additionally available between a user and a computer, without departing from the scope of the present invention.

In another embodiment the method and system of the present invention may be used for the management of thyroid diseases. For example, a drug such as thyroxine may be packaged in a dispenser such as a blister pack or circular dial pack with a child resistant housing or closure and a MEMSO® monitor. Alternatively, a stackable magazine like dispenser with pills in a size and shape for that dispenser may be used.

Monitoring and patient prompts may be utilized to individualize dosing and therapy when used in conjunction with measurement of a patient's thyroid hormone level.

Use of such a system will encourage improved patient compliance or permit the modification of dose in view of the patient's compliance history.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

What is claimed:

1. A method of individualizing the treatment of a patient associated with a prescribed drug regimen comprising the steps of:
developing a pharmacokinetic model, which predicts the drug concentration over time in the patient in response to the drug dosage history of the patient;
developing a pharmacodynamic model, which includes a predicted level of effectiveness for various levels of dosing and various degrees of deviation from prescribed dosing regimen;
prescribing a drug regimen for the patient, designed to achieve a desired pharmacological effect, based upon the pharmacokinetic model and the pharmacodynamic model;
monitoring the adherence of the patient to a prescribed dosing regimen including the degree of deviation;
measuring the pharmacological effect in the patient of the prescribed dosing regimen;
comparing the measured effect with the level of effectiveness, predicted by the pharmacodynamic model, after taking into account the adherence of the patient to the prescribed dosing regimen; and
if the measured effect deviates from the predicted level of effectiveness,
performing additional tests to determine the actual drug concentration over time in the particular patient,
adjusting the pharmacokinetic model based upon the actual determined drug concentration over time, and
adjusting the prescribed drug regimen for the patient to account for adjustments in the pharmacokinetic model.

2. The method of claim 1, wherein the additional tests include therapeutic drug monitoring.

3. The method of claim 2, wherein the drug regimen includes an antiretroviral agent for treating a viral infection, and wherein if the results of the therapeutic drug monitoring test indicates that the drug concentration over time is within the expected range, then performing drug resistance testing to determine the presence or the emergence of a drug resistant virus.

4. The method of claim 3, wherein if a drug resistant virus is present or has emerged, adjusting the prescribed drug regimen to include another antiretroviral agent.

5. The method of claim 1, wherein the drug regimen includes an antiretroviral agent for treating a viral infection, and wherein the pharmacological effect includes at least one of a change in the viral load and an emergence of a resistant strain.

6. The method of claim 1, wherein the drug concentration over time in the patient includes the drug concentration over time in the plasma of the patient.

7. The method of claim 1, wherein the degree of deviation from the prescribed dosing regimen includes a measure of the number of missed doses.

8. The method of claim 1, wherein the degree of deviation from the prescribed dosing regimen includes a measure of the frequency of missed doses.

9. The method of claim 1, wherein when a dose is missed or delayed, the degree of deviation from the prescribed dosing regimen takes into account the length in time between the delayed dose or the dose taken after the preceding dose was missed, and the preceding dose, which was taken.

10. The method of claim 1, wherein monitoring the adherence of the patient to a prescribed dosing regimen includes monitoring the time and date the patient accesses an enclosure containing each of one or more medications prescribed as part of the prescribed dosing regimen.

11. The method of claim 1, wherein the pharmacokinetic model takes into account the rates of at least one of drug absorption, drug distribution, drug metabolism, and drug excretion.

12. A method of designing a clinical trial, which determines the effectiveness of a drug in producing a desired pharmacological effect over abroad range of patient adherence to a prescribed drug regimen comprising the steps of:
prescribing a drug regimen for one or more patients;
monitoring the adherence of the one or more patients to the prescribed drug regimen;
measuring the pharmacological effect in the one or more patients at various intervals; and
relating the measured pharmacological effect to the patient adherence and the degree of deviation from the prescribed dosing regimen, if any.

13. The method of claim 12 wherein the decision to measure the pharmacological effect in the one or more patients, in at least some instances, is prompted to occur, when the adherence to the drug regimen of the one or more patients is at a point in which the results of the testing is not substantially covered by already existing test data.

14. The method of claim 13, wherein the results of the testing not being covered by already existing test data includes instances where the additional data would reduce the degree of any interpolation required in making a prediction of the pharmacological effect from the already existing data.

15. A method of providing patient care comprising:
prescribing a drug regimen for a patient directed to achieving and maintaining a predetermined level of wellness, when a predetermined level of patient compliance is met or exceeded;
monitoring the adherence of the patient to the prescribed drug regimen;
if the patient compliance level approaches or falls below the predetermined level of patient compliance,
comparing the cost of subsequent corrective action in drug dosing, if necessary, to compensate for non-compliant patient behavior with the cost of intervening with the patient to encourage patient compliance,
if it is more cost effective to compensate for non-compliant patient behavior by adjusting the drug regimen, than by adjusting the drug regimen, and
if it is equally or more cost effective to intervene with the patient to encourage patient compliance, then intervening with the patient; and
if the patient compliance level meets or exceeds the predetermined level of patient compliance, and the predetermined level of wellness fails to be achieved and maintained, determining the need for change in the prescribed drug regimen.

16. The method of claim 15 wherein determining the need for change in the prescribed drug regimen includes
testing the patient to determine the drug concentration over time in the patient,
if the determined drug concentration over time in the patient deviates from the expected pattern, then adjusting at least one of the frequency and the dosing levels of the prescribed drug regimen to compensate for the determined deviation in the expected pattern of the drug concentration over time, and
if the determined time course of drug concentration in the patient is consistent with the expected pattern, altering the prescribed drug regimen to include alternative therapies.

17. The method of claim 15 wherein intervening with the patient includes explaining the consequences of sub-optimal compliance.

18. The method of claim 15, wherein monitoring the adherence of the patient to a prescribed dosing regimen includes monitoring the time and date the patient accesses an enclosure containing each of one or more medications prescribed as part of the prescribed dosing regimen.

19. The method of claim 18 wherein intervening with the patient includes contacting the patient about a missed event from prescribed drug regimen after a predetermined period of time, and the event has still not occurred.

20. A system for managing a patient treatment program comprising:
one or more enclosures each containing medication subscribed as part of a prescribed drug regimen for a respective one of the one or more patients, each enclosure being adapted for monitoring the access of the respective patient to the prescribed medication; and
a patient health management computer comprising
a communication unit for receiving the access information from each of the one or more enclosures,
a processor for executing a plurality of prestored instructions and data including
instructions and data for creating and maintaining a pharmacokinetic model, which predicts the drug concentration over time in the patients, based at least in part upon the access information received from the corresponding enclosures,
instructions and data for creating and maintaining a pharmacodynamic model, which receives the drug concentration over time predicted by the pharmacokinetic model, and predicts the level of effectiveness for various levels of dosing and various degrees of deviation from the prescribed dosing regimen, and
instructions and data for creating and maintaining a decision analytic model, which receives the dosing history, the predicted drug concentration over time in the respective patients, and the predicted levels of effectiveness, for recommending when at least one of one or more tests should be performed on a patient to determine the actual condition of at least one aspect of the patient, and for deciding when the prescribed dosing regimen should be changed, and
an interface unit for communicating to a user the recommendation when a test should be performed, and for receiving the results of the tests performed.

21. The system of claim 20, wherein the enclosure includes a reservoir encapsulating a space capable of holding one or more doses of a medication, said reservoir having an opening through which access to the one or more doses is possible, and a cap, which selectively covers said opening, and which is adapted to detect its position relative to the reservoir between covering said opening and not covering said opening.

22. The system of claim 21, wherein the cap includes a calender and clock for detecting when the patient accesses the enclosure.

23. The system of claim 21, wherein the cap includes a memory for storing the access data for each instance that the patient accesses the enclosure.

24. The system of claim 21, wherein the cap includes a transmitter and a receiver for wirelessly communicating the access data with the communication unit of the computer data server.

25. The system of claim 20, wherein the system is used for treating a patient with a viral infection, and the pharmacodynamic model includes a submodel for predicting the emergence of a drug resistant strain.

26. The system of claim 20, wherein the system is used for treating a patient with a viral infection, and the pharmacodynamic model includes a submodel for predicting the response of CD4 cell counts to the prescribed drug regimen.

27. The system of claim 20, wherein the prescribed drug regimen includes the use of an antiretroviral agent.

28. The system of claim 20, wherein the one or more tests include at least one of therapeutic drug monitoring for measuring the actual drug concentrations over time in the patient, a viral load test for measuring the actual amount of virus present in the patient, a drug resistance test for detecting the emergence of a drug resistant strain of the virus and measuring the relative amount of the drug resistant strain, and a CD4 count for measuring the absolute and relative amounts of CD4 cells in the plasma of the patient.

29. A method for managing an antiretroviral treatment program of a patient including one or more drugs comprising the steps of:
prescribing a drug regimen for treating a viral infection, the severity of which is represented by a viral load;
if the condition of the patient, including the viral load, does not improve after a predetermined period of time
monitoring the adherence of the patient to the prescribed drug regimen including a dosing history,
executing a pharmacokinetic model, which receives the dosing history and predicts the drug concentration over time in the patient,
executing a pharmacodynamic mode, which receives the drug concentration over time from the pharmacokinetic model and predicts the likelihood of emergence of a drug resistant virus, and
executing a decision analytic model, which receives the dosing history, the drug concentration over time, and the likelihood of emergence of a drug resistant virus and determines when at least one of additional tests should be performed and when the prescribed drug regimen should be altered.

30. The method of claim 29 wherein the dosing history includes both the time and day each of the drug doses is taken.

31. The method of claim 30 wherein monitoring the adherence of the patient includes determining the degree of deviation by comparing the actual time a particular drug dose is taken with the scheduled time the particular drug dose is taken.

32. The method of claim 29 wherein the pharmacodynamic model includes determining the instances, if any, and the corresponding duration in which the level of drug concentration in the patient, predicted by the pharmacokinetic model, falls below a predetermined level.

33. The method of claim 29 wherein the decision analytic model includes an economic submodel, which takes into account the economic costs of the additional tests and the predicted likelihood that the tests will produce useful information.

34. The method of claim 29 wherein the additional tests include therapeutic drug monitoring to measure the actual drug concentration over time for comparing with the drug concentration over time predicted by the pharmacokinetic model.

35. The method of claim 34 wherein if the actual drug concentration over time substantially deviates from the predicted drug concentration over time, then adjusting the pharmacokinetic model to account for the deviation.

36. The method of claim 35 wherein the predicted drug concentration over time includes a range of predicted drug concentration values, which varies over time.

37. The method of claim 29 wherein the additional tests include viral load testing to measure the actual amount of virus present for comparing with previously measured amounts of the virus present and for determining a change, if any, in the amount of virus present.

38. The method of claim 37 wherein if the viral load testing identifies a change in the amount of virus present, which corresponds to an increase in the amount of virus present, and the increase is inconsistent with the amount predicted given the monitored adherence of the patient to the prescribed drug regimen and the corresponding predicted drug concentration over time, then testing for the presence or emergence of drug resistant virus.

39. The method of claim 29 wherein the additional tests include drug resistance testing to test for the emergence of drug resistant virus, and the amount of drug resistant virus present, if any.

40. The method of claim 39 wherein if the drug resistance testing identifies the presence or emergence of drug resistant virus, then adjusting the prescribed drug regimen.

41. The method of claim 29 wherein the pharmacodynamic model includes a submodel for predicting the response of CD4 cell counts, based upon the received value of the drug concentration over time in the patient.

42. The method of claim 41 wherein the additional tests include a test which measures CD4 cell counts for determining the overall health of the immune system.

43. The method of claim 42 wherein if the test which measure CD4 cell counts falls below a predetermined threshold, then adjusting prescribed drug regimen to include drugs for preventing opportunistic infections.

44. The method of claim 1 wherein the drug regimen is for the treatment of thyroid disease.

45. The system of claim 20 wherein the system is used for treating a patient with thyroid disease.
